(19) United States
(12) Patent Application Publication
(10) Pub. No.: US 2022/0370423 A1
(43) Pub. Date: Nov. 24, 2022

(54) USE OF 1-[4-BROMO-5-[1-ETHYL-7-(METHYLAMINO)-2-OXO-1,2-DIHYDRO-1,6-NAPHTHYRIDIN-3-YL]-2-FLUOROPHENYL]-3-PHENYLUREA AND ANALOGS FOR THE TREATMENT OF CANCERS ASSOCIATED WITH GENETIC ABNORMALITIES IN PLATELET DERIVED GROWTH FACTOR RECEPTOR ALPHA

(71) Applicant: Deciphera Pharmaceuticals, LLC, Waltham, MA (US)

(72) Inventors: Daniel L. Flynn, Waltham, MA (US); Michael D. Kaufman, Waltham, MA (US); Oliver Rosen, Waltham, MA (US); Bryan D. Smith, Waltham, MA (US)

(21) Appl. No.: 17/845,275

(22) Filed: Jun. 21, 2022

Related U.S. Application Data
(63) Continuation of application No. 16/617,721, filed on Nov. 27, 2019, now abandoned.

(51) Int. Cl. A61K 31/4375; A61P 35/04; A61K 31/495

(52) U.S. Cl. CPC A61K 31/4375; A61P 35/04; A61K 31/495

(57) ABSTRACT

The present disclosure relates to the use of 1-[4-bromo-5-[1-ethyl-7-(methylamino)-2-oxo-1,2-dihydro-1,6-naphthyridin-3-yl]-2-fluorophenyl]-3-phenylurea or 1-(5-(7-amino-1-ethyl-2-oxo-1,2-dihydro-1,6-naphthyridin-3-yl)-4-bromo-2-fluorophenyl)-3-phenylurea in the treatment of cancers. Specifically, the disclosure is directed to methods of inhibiting PDGFR kinases and treating cancers and disorders associated with inhibition of PDGFR kinases including lung adenocarcinoma, squamous cell lung cancer, glioblastoma, pediatric glioma, astrocytomas, sarcomas, gastrointestinal stromal tumors, malignant peripheral nerve sheath sarcoma, intimal sarcomas, hypereosinophilic syndrome, idiopathic hypereosinophilic syndrome, chronic eosinophilic leukemia, eosinophilia-associated acute myeloid leukemia, or lymphoblastic T-cell lymphoma.

### CROSS-REFERENCE TO RELATED APPLICATIONS

This application is a continuation of U.S. application Ser. No. 16/617,721 filed Nov. 27, 2019, which is a National Stage Entry of International Application Number PCT/US2017/035005 filed May 30, 2017 under 35 U.S.C. § 371, the contents of each of which are incorporated herein by reference in their entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: DECP_073_00US_SeqList_ST25.txt, date recorded: May 30, 2017, file size 24 kilobytes).

### FIELD OF INVENTION

The present disclosure relates to the use of 1-[4-bromo-5-[1-ethyl-7-(methylamino)-2-oxo-1,2-dihydro-1,6-naphthyridin-3-yl]-2-fluorophenyl]-3-phenylurea or 1-(5-(7-amino-1-ethyl-2-oxo-1,2-dihydro-1,6-naphthyridin-3-yl)-4-bromo-2-fluorophenyl)-3-phenylurea in the treatment of cancers. Specifically, the disclosure is directed to methods of inhibiting PDGFR kinases and treating cancers and disorders associated with inhibition of PDGFR kinases including lung adenocarcinoma, squamous cell lung cancer, glioblastoma, pediatric glioma, astrocytomas, sarcomas, gastrointestinal stromal tumors (GISTs), malignant peripheral nerve sheath sarcoma, intimal sarcomas, hypereosinophilic syndrome, eosinophilia-associated acute myeloid leukemia, idiopathic hypereosinophilic syndrome, chronic eosinophilic leukemia or lymphoblastic T-cell lymphoma.

### BACKGROUND OF THE INVENTION

Oncogenic genomic alterations of PDGFRα kinase or overexpression of PDGFRα kinase have been shown to be causative of human cancers.

Missense mutations of PDGFRα kinase have been shown to be causative of a subset of GISTs. PDGFRα mutations are oncogenic drivers in approximately 8-10% of GISTs (Corless, Modern Pathology 2014; 27:S1-16). The predominant PDGFRα mutation is exon 18 D842V, although other exon 18 mutations including D846Y, N848K, and Y849K, and exon 18 insertion-deletion mutations (INDELs) including RD841-842KI, DI842-843-IM, and HDSN845-848P have also been reported. Furthermore, rare mutations in PDGFRα exons 12 and 14 have also been reported (Corless et al, J. Clinical Oncology 2005; 23:5357-64).

The PDGFRα exon 18 deletion mutations AD842-H845 and AI843-D846 have been reported in GIST (Lasota et al, Laboratory Investigation 2004; 84:874-83).

Amplification or mutations of PDGRFα have been described in human tissues of malignant peripheral nerve sheath tumors (MPNST) (Holtkamp et al, Carcinogenesis 2006; 27:664-71).

Amplification of PDGFRα has been described in multiple skin lesions of undifferentiated pleomorphic sarcoma (Osio et al, J. Cutan Pathol 2017; 44:477-79) and in intimal sarcoma (Zhao et al, Genes Chromosomes and Cancer, 2002; 34: 48-57; Dewaele et al, Cancer Res 2010; 70: 7304-14).

Amplification of PDGFRα has been linked to a subset of lung cancer patients. 4q12, containing the PDGFRα gene locus, is amplified in 3-7% of lung adenocarcinomas and 8-10% of lung squamous cell carcinomas (Ramos et al, Cancer Biol Ther. 2009; 8: 2042-50; Heist et al, J Thorac Oncol. 2012; 7: 924-33).

Mutations in the IDH protein produce a new onco-metabolite, 2-hydroxyglutarate, which interferes with iron-dependent hydroxylases, including the TET family of 5′-methylcytosine hydroxylases. TET enzymes catalyze a key step in the removal of DNA methylation. Flavahan et al demonstrated that human IDH mutant gliomas exhibit hypermethylation at DNA cohesin and CCCTC-binding factor (CTCF)-binding sites, compromising binding of this methylation-sensitive insulator protein (Flavahan et al., Nature 2016; 529:110). Reduced CTCF binding is associated with loss of insulation between topological domains and aberrant gene activation. Specifically, loss of CTCF at a domain boundary permits a constitutive enhancer to interact aberrantly with the receptor tyrosine kinase gene PDGFRA, a prominent glioma oncogene. Thus, IDH mutated cancers can be predisposed to mediate oncogenic events through activation and overexpression of wild type PDGFRα.

PDGFRα amplification is common in pediatric and adult high-grade astrocytomas and identified a poor prognostic group in IDH1 mutant glioblastoma. PDGFRα amplification was frequent in pediatric (29.3%) and adult (20.9%) tumors. PDGFRα amplification was reported to increase with grade and in particular to be associated with a less favorable prognosis in IDH1 mutant de novo GBMs (Phillips et al, Brain Pathology, 2013; 23:565-73).

The PDGFRα locus in PDGFRα-amplified gliomas has been demonstrated to present a PDGFRα exon 8,9 intragenic deletion rearrangement. This intragenic deletion was common, being present in 40% of the glioblastoma multiformes (GBMs) presenting with PDGFRα amplification. Tumors with this rearrangement displayed histologic features of oligodendroglioma, and the PDGFRα exon 8,9 intragenic deletion showed constitutively elevated tyrosine kinase activity (Ozawa et al, Genes and Development 2010; 24:2205-18).

The FIP1L1-PDGFRA fusion protein is oncogenic in a subset of patients with hypereosinophilic syndrome (Elling et al, Blood 2011; 117; 2935). FIPIL1-PDGFRα fusion has also been identified in eosinophilia-associated acute myeloid leukemia and lymphoblastic T-cell lymphoma (Metzgeroth et al, Leukemia 2007; 21:1183-88).

In summary, mutations, deletions, rearrangements, and amplification of the PDGFRα gene are linked to a number of solid and hematological cancers. Given the complex function of the PDGRFα gene and the potential utility for PDGFRα inhibitors in the treatment of various solid and hematological cancers, there is a need for inhibitors with good therapeutic properties.

### SUMMARY OF THE INVENTION

One aspect of the invention relates to a method of treating or preventing a PDGFR kinase-mediated tumor growth or tumor progression comprising administering to a patient in need thereof an effective amount of 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof.

Another aspect of the invention is directed to a method of inhibiting PDGFR kinase comprising administering to a patient in need thereof an effective amount of 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof.

Another aspect of the invention relates to a method of inhibiting a PDGFR kinase or treating a PDGFR kinase-mediated tumor growth or tumor progression. The method comprises administering to a patient in need thereof 1-[4-bromo-5-[l-ethyl-7-(methylamino)-2-oxo-1,2-dihydro-1,6-naphthyridin-3-yl]-2-fluorophenyl]-3-phenylurea, or a pharmaceutically acceptable salt thereof as a single agent or in combination with other cancer targeted therapeutic agents, cancer-targeted biologicals, immune checkpoint inhibitors, or chemotherapeutic agents.

Yet another aspect of the invention provides a method of treating glioblastoma, comprising administering to a patient in need thereof an effective amount of 1-[4-bromo-5-[1-ethyl-7-(methylamino)-2-oxo-1,2-dihydro-1,6-naphthyridin-3-yl]-2-fluorophenyl]-3-phenylurea, or a pharmaceutically acceptable salt thereof.

Another aspect of the invention relates to a method of treating PDGFRα-mediated gastrointestinal stromal tumors, comprising administering to a patient in need thereof an effective amount of 1-[4-bromo-5-[l-ethyl-7-(methylamino)-2-oxo-1,2-dihydro-1,6-naphthyridin-3-yl]-2-fluorophenyl]-3-phenylurea, or a pharmaceutically acceptable salt thereof.

Another aspect of the invention relates to a method of treating or preventing a PDGFR kinase-mediated tumor growth or tumor progression comprising administering to a patient in need thereof an effective amount of 1-(5-(7-amino-1-ethyl-2-oxo-1,2-dihydro-1,6-naphthyridin-3-yl)-4-bromo-2-fluorophenyl)-3-phenylurea, or a pharmaceutically acceptable salt thereof.

Another aspect of the invention relates to a method of inhibiting PDGFR kinase, comprising administering to a patient in need thereof an effective amount of 1-(5-(7-amino-1-ethyl-2-oxo-1,2-dihydro-1,6-naphthyridin-3-yl)-4-bromo-2-fluorophenyl)-3-phenylurea, or a pharmaceutically acceptable salt thereof.

Another aspect of the invention relates to a method of inhibiting a PDGFR kinase or treating a PDGFR kinase-mediated tumor growth or tumor progression. The method comprises administering to a patient in need thereof 1-(5-(7-amino-1-ethyl-2-oxo-1,2-dihydro-1,6-naphthyridin-3-yl)-4-bromo-2-fluorophenyl)-3-phenylurea, or a pharmaceutically acceptable salt thereof as a single agent or in combination with other cancer targeted therapeutic agents, cancer-targeted biologicals, immune checkpoint inhibitors, or chemotherapeutic agents.

Yet another aspect of the invention provides a method of treating glioblastoma, comprising administering to a patient in need thereof an effective amount of 1-(5-(7-amino-1-ethyl-2-oxo-1,2-dihydro-1,6-naphthyridin-3-yl)-4-bromo-2-fluorophenyl)-3-phenylurea, or a pharmaceutically acceptable salt thereof.

Another aspect of the invention relates to a method of treating PDGFRα-mediated gastrointestinal stromal tumors, comprising administering to a patient in need thereof an effective amount of 1-(5-(7-amino-1-ethyl-2-oxo-1,2-dihydro-1,6-naphthyridin-3-yl)-4-bromo-2-fluorophenyl)-3-phenylurea, or a pharmaceutically acceptable salt thereof.

Another aspect of the invention relates to the in vivo biosynthetic formation of 1-(5-(7-amino-1-ethyl-2-oxo-1,2-dihydro-1,6-naphthyridin-3-yl)-4-bromo-2-fluorophenyl)-3-phenylurea (Compound B) after oral administration of 1-[4-bromo-5-[1-ethyl-7-(methylamino)-2-oxo-1,2-dihydro-1,6-naphthyridin-3-yl]-2-fluorophenyl]-3-phenylurea (Compound A).

The present disclosure further provides methods of inhibiting PDGFR kinases and treating cancers and disorders associated with inhibition of PDGFR kinases including lung adenocarcinoma, squamous cell lung cancer, glioblastoma, pediatric glioma, astrocytomas, sarcomas, gastrointestinal stromal tumors, malignant peripheral nerve sheath sarcoma, intimal sarcomas, hypereosinophilic syndrome, idiopathic hypereosinophilic syndrome, chronic eosinophilic leukemia, eosinophilia-associated acute myeloid leukemia, or lymphoblastic T-cell lymphoma.

The invention also provides methods of inhibiting PDGFRα kinase, oncogenic PDGFRα missense mutations, oncogenic deletion PDGFRα mutations, oncogenic PDGFRα gene rearrangements leading to PDGFRα fusion proteins, or oncogenic PDGFRα gene amplification.

The invention also provides methods of use of 1-[4-bromo-5-[1-ethyl-7-(methylamino)-2-oxo-1,2-dihydro-1,6-naphthyridin-3-yl]-2-fluorophenyl]-3-phenylurea or 1-(5-(7-amino-1-ethyl-2-oxo-1,2-dihydro-1,6-naphthyridin-3-yl)-4-bromo-2-fluorophenyl)-3-phenylurea.

### BRIEF DESCRIPTION OF THE DRAWINGS

FIG. 1A illustrates an MRI scan of the brain of a patient with glioblastoma tumor exhibiting PDGFRα amplification at baseline. FIG. 1B shows proof of the tumor reduction after at cycle 9. FIG. 1C shows an MRI scan of the same brain after cycle 12.

### DETAILED DESCRIPTION OF THE INVENTION

It has been found that 1-[4-bromo-5-[1-ethyl-7-(methylamino)-2-oxo-1,2-dihydro-1,6-naphthyridin-3-yl]-2-fluorophenyl]-3-phenylurea (Compound A) and 1-(5-(7-amino-1-ethyl-2-oxo-1,2-dihydro-1,6-naphthyridin-3-yl)-4-bromo-2-fluorophenyl)-3-phenylurea (Compound B) unexpectedly inhibit wild-type and oncogenic protein forms of PDGFR kinases. The present invention provides a method for treating cancer by inhibiting oncogenic PDGFRα kinase-mediated tumor growth or tumor progression comprising administering to a patient in need thereof an effective amount of 1-[4-bromo-5-[l-ethyl-7-(methylamino)-2-oxo-1,2-dihydro-1,6-naphthyridin-3-yl]-2-fluorophenyl]-3-phenylurea, 1-(5-(7-amino-1-ethyl-2-oxo-1,2-dihydro-1,6-naphthyridin-3-yl)-4-bromo-2-fluorophenyl)-3-phenylurea, or a pharmaceutically acceptable salt thereof.

### Definition

Compounds A and B as used herein refers to 1-[4-bromo-5-[1-ethyl-7-(methylamino)-2-oxo-1,2-dihydro-1,6-naphthyridin-3-yl]-2-fluorophenyl]-3-phenylurea and 1-(5-(7-amino-1-ethyl-2-oxo-1,2-dihydro-1,6-naphthyridin-3-yl)-4-bromo-2-fluorophenyl)-3-phenylurea. Pharmaceutically acceptable salts, tautomers, hydrates, and solvates, of Compounds A and B are also contemplated in this disclosure. The structures of Compounds A and B are represented below:

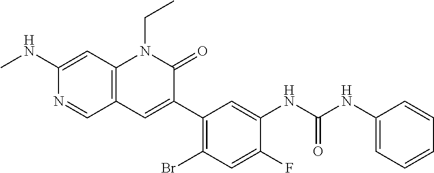 [SMILES: CCn1c(=O)c(-c2cc(NC(=O)Nc3ccccc3)c(F)cc2Br)cc2cnc(NC)cc21]

### 1-[4-bromo-5-[1-ethyl-7-(methylamino)-2-oxo-1,2-dihydro-1,6-naphthyridin-3-yl]-2-fluorophenyl]-3-phenylurea (Compound A)

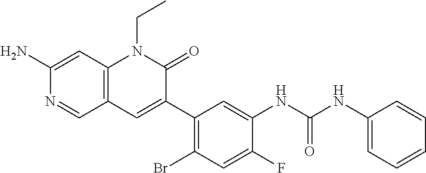 [SMILES: CCn1c(=O)c(-c2cc(NC(=O)Nc3ccccc3)c(F)cc2Br)cc2cnc(N)cc21]

### 1-(5-(7-amino-1-ethyl-2-oxo-1,2-dihydro-1,6-naphthyridin-3-yl)-4-bromo-2-fluorophenyl)-3-phenylurea (Compound B)

Methods of making Compound A and Compound B are disclosed in U.S. Pat. No. 8,461,179B1 the contents of which are incorporated herein by reference. The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs. The initial definition provided for a group or term provided in this disclosure applies to that group or term throughout the present disclosure individually or as part of another group, unless otherwise indicated.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals. “Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

Subjects or patients “in need of treatment” with a compound of the present disclosure, or patients “in need of PDGFRα inhibition” include patients with diseases and/or conditions that can be treated with the compounds of the present disclosure to achieve a beneficial therapeutic result. A beneficial outcome includes an objective response, increased progression free survival, increased survival, prolongation of stable disease, and/or a decrease in the severity of symptoms or delay in the onset of symptoms. For example, a patient in need of treatment is suffering from a tumor growth or tumor progression; the patient is suffering from, but not limited to, lung adenocarcinoma, squamous cell lung cancer, glioblastoma, pediatric glioma, astrocytomas, sarcomas, gastrointestinal stromal tumors, malignant peripheral nerve sheath sarcoma, intimal sarcomas, hypereosinophilic syndrome, idiopathic hypereosinophilic syndrome, chronic eosinophilic leukemia, eosinophilia-associated acute myeloid leukemia, or lymphoblastic T-cell lymphoma and the like.

As used herein, an “effective amount” (or “pharmaceutically effective amount”) of a compound disclosed herein, is a quantity that results in a beneficial clinical outcome of the condition being treated with the compound compared with the absence of treatment. The amount of the compound or compounds administered will depend on the degree, severity, and type of the disease or condition, the amount of therapy desired, and the release characteristics of the pharmaceutical formulation. It will also depend on the subject's health, size, weight, age, sex and tolerance to drugs. Typically, the compound is administered for a sufficient period of time to achieve the desired therapeutic effect.

The terms “treatment,” “treat,” and “treating,” are meant to include the full spectrum of intervention in patients with “cancer” with the intention to prevent tumor growth from which the patient is suffering and/or to prevent tumor progression on a given treatment, such as administration of the active compound to alleviate, slow or reverse one or more of the symptoms and to delay progression of the cancer even if the cancer is not actually eliminated. Treating can be curing, improving, or at least partially ameliorating the disorder.

“Cancer” as defined herein refers to a new growth which has the ability to invade surrounding tissues, metastasize (spread to other organs) and which may eventually lead to the patient's death if untreated. “Cancer” can be a solid tumor or a liquid tumor.

“Tumor” as used herein refers to a mass. This is a term that may refer to benign (generally harmless) or malignant (cancerous) growths. Malignant growth can originate from a solid organ or the bone marrow. The latter is often referred to as liquid tumors.

“Tumor growth” as defined herein refers to growth of a mass caused by genomic alterations of the PDGFRα kinase.

“Tumor progression” as defined herein refers to tumor growth of an existing PDGFRα-dependent tumor wherein such tumor growth of an existing mass is caused by further genomic alterations of the PDGFRα kinase resistant to a treatment.

One aspect of the invention relates to a method of treating or preventing a PDGFR kinase-mediated tumor growth or tumor progression comprising administering to a patient in need thereof an effective amount of 1-[4-bromo-5-[l-ethyl-7-(methylamino)-2-oxo-1,2-dihydro-1,6-naphthyridin-3-yl]-2-fluorophenyl]-3-phenylurea (Compound A), or a pharmaceutically acceptable salt thereof.

In one embodiment, Compound A or a pharmaceutically acceptable salt thereof is administered to a cancer patient wherein tumor growth or tumor progression is caused by PDGFRα kinase overexpression, oncogenic PDGFRα missense mutations, oncogenic deletion PDGFRα mutations, oncogenic PDGFRα gene rearrangements leading to PDGFRα fusion proteins, PDGFRα intragenic in-frame deletions, and/or oncogenic PDGFRα gene amplification. In one embodiment, the tumor growth or tumor progression is caused by PDGFRα kinase overexpression. In another embodiment, the tumor growth or tumor progression is caused by oncogenic PDGFRα missense mutations. In another embodiment, the tumor growth or tumor progression is caused by oncogenic deletion PDGFRα mutations. In another embodiment, the tumor growth or tumor progression is caused by oncogenic PDGFRα gene rearrangements leading to PDGFRα fusion proteins. In another embodiment, the tumor growth or tumor progression is caused by PDGFRα intragenic in-frame deletions. In another embodiment, the tumor growth or tumor progression is caused by oncogenic PDGFRα gene amplification.

In another embodiment, Compound A or a pharmaceutically acceptable salt thereof is administered to a cancer patient wherein tumor growth or tumor progression is caused by D842V mutant PDGFRα, V561D mutant PDGFRα, exon 18 PDGFRα deletion mutations including 842-845 deletion mutant PDGFRα, exon 8,9 PDGFRα in-frame deletion mutation, PDGFRα fusions including FIPIL1-PDGFRα, or PDGFRα amplification.

In another embodiment, Compound A or a pharmaceutically acceptable salt thereof is administered to a cancer patient wherein the cancer is lung adenocarcinoma, squamous cell lung cancer, glioblastoma, pediatric glioma, astrocytomas, sarcomas, gastrointestinal stromal tumors, malignant peripheral nerve sheath sarcoma, intimal sarcomas, hypereosinophilic syndrome, idiopathic hypereosinophilic syndrome, chronic eosinophilic leukemia, eosinophilia-associated acute myeloid leukemia, or lymphoblastic T-cell lymphoma. In one embodiment, the cancer is glioblastoma. In another embodiment, the cancer is a gastrointestinal stromal tumor.

In another embodiment, Compound A or a pharmaceutically acceptable salt thereof is administered to a cancer patient as a single agent or in combination with other cancer targeted therapeutic agents, cancer-targeted biologicals, immune checkpoint inhibitors, or chemotherapeutic agents.

Another aspect of the invention relates to a method of treating or preventing a PDGFR kinase-mediated tumor growth or tumor progression comprising administering to a patient in need thereof an effective amount of 1-(5-(7-amino-1-ethyl-2-oxo-1,2-dihydro-1,6-naphthyridin-3-yl)-4-bromo-2-fluorophenyl)-3-phenylurea (Compound B), or a pharmaceutically acceptable salt thereof.

In one embodiment, Compound B or a pharmaceutically acceptable salt thereof is administered to a cancer patient wherein tumor growth or tumor progression is caused by PDGFRα kinase overexpression, oncogenic PDGFRα missense mutations, oncogenic deletion PDGFRα mutations, oncogenic PDGFRα gene rearrangements leading to PDGFRα fusion proteins, PDGFRα intragenic in-frame deletions, and/or oncogenic PDGFRα gene amplification. In one embodiment, the tumor growth or tumor progression is caused by PDGFRα kinase overexpression. In another embodiment, the tumor growth or tumor progression is caused by oncogenic PDGFRα missense mutations. In another embodiment, the tumor growth or tumor progression is caused by oncogenic deletion PDGFRα mutations. In another embodiment, the tumor growth or tumor progression is caused by oncogenic PDGFRα gene rearrangements leading to PDGFRα fusion proteins. In another embodiment, the tumor growth or tumor progression is caused by PDGFRα intragenic in-frame deletions. In another embodiment, the tumor growth or tumor progression is caused by oncogenic PDGFRα gene amplification.

In another embodiment, Compound B or a pharmaceutically acceptable salt thereof is administered to a cancer patient wherein tumor growth or tumor progression is caused by D842V mutant PDGFRα, V561D mutant PDGFRα, exon 18 PDGFRα deletion mutations including 842-845 deletion mutant PDGFRα, exon 8,9 PDGFRα in-frame deletion mutation, PDGFRα fusions including FIPIL1-PDGFRα, or PDGFRα amplification.

In another embodiment, Compound B or a pharmaceutically acceptable salt thereof is administered to a cancer patient wherein the cancer is lung adenocarcinoma, squamous cell lung cancer, glioblastoma, pediatric glioma, astrocytomas, sarcomas, gastrointestinal stromal tumors, malignant peripheral nerve sheath sarcoma, intimal sarcomas, hypereosinophilic syndrome, idiopathic hypereosinophilic syndrome, chronic eosinophilic leukemia, eosinophilia-associated acute myeloid leukemia, or lymphoblastic T-cell lymphoma. In one embodiment, the cancer is glioblastoma. In another embodiment, the cancer is a gastrointestinal stromal tumor. In another embodiment, Compound B or a pharmaceutically acceptable salt thereof is administered to a cancer patient as a single agent or in combination with other cancer targeted therapeutic agents, cancer-targeted biologicals, immune checkpoint inhibitors, or chemotherapeutic agents.

### Pharmaceutical Compositions and Methods of Treatment

It is further noted that the present disclosure is directed to methods of treatment involving the administration of the compound of the present disclosure, or a pharmaceutical composition comprising such a compound. The pharmaceutical composition or preparation described herein may be used in accordance with the present disclosure for the treatment of various cancers including lung adenocarcinoma, squamous cell lung cancer, glioblastoma, pediatric glioma, astrocytomas, sarcomas, gastrointestinal stromal tumors, malignant peripheral nerve sheath sarcoma, intimal sarcomas, hypereosinophilic syndrome, idiopathic hypereosinophilic syndrome, chronic eosinophilic leukemia, eosinophilia-associated acute myeloid leukemia, or lymphoblastic T-cell lymphoma.

The compounds utilized in the treatment methods of the present disclosure, as well as the pharmaceutical compositions comprising them, may accordingly be administered alone, or as part of a treatment protocol or regiment that includes the administration or use of other beneficial compounds (as further detailed elsewhere herein).

In some embodiments the present invention relates to a method of using a pharmaceutical composition comprising compound A or B and a pharmaceutically acceptable carrier comprising one or more additional therapeutic agents. The additional therapeutic agents include, but are not limited to, cytotoxic agent, cisplatin, doxorubicin, etoposide, irinotecan, topotecan, paclitaxel, docetaxel, the epothilones, tamoxifen, 5-fluorouracil, methotrexate, temozolomide, cyclophosphamide, lonafarib, tipifarnib, 4-((5-((4-(3-chlorophenyl)-3-oxopiperazin-1-yl)methyl)-1H-imidazol-1-yl)methyl)benzonitrile hydrochloride, (R)-1-((1H-imidazol-5-yl)methyl)-3-benzyl-4-(thiophen-2-ylsulfonyl)-2,3,4,5-tetrahydro-1H-benzo diazepine-7-carbonitrile, cetuximab, imatinib, interferon alfa-2b, pegylated interferon alfa-2b, aromatase combinations, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, leucovorin, oxaliplatin, pentostatine, vinblastine, vincristine, vindesine, bleomycin, dactinomycin, daunorubicin, epirubicin, idarubicin, mithramycin, deoxycoformycin, mitomycin-C, L-asparaginase, teniposide 17α-ethinyl estradiol, diethylstilbestrol, testosterone, prednisone, fluoxymesterone, dromostanolone propionate, testolactone, megestrol acetate, methylprednisolone, methyltestosterone, prednisolone, triamcinolone, chlorotrianisene, 17α-hydroxyprogesterone, aminoglutethimide, estramustine, medroxyprogesterone acetate, leuprolide acetate, flutamide, toremifene citrate, goserelin acetate, carboplatin, hydroxyurea, amsacrine, procarbazine, mitotane, mitoxantrone, levamisole, vinorelbine, anastrazole, letrozole, capecitabine, raloxifene, droloxafine, hexamethylmelamine, bevacizumab, trastuzumab, tositumomab, bortezomib, ibritumomab tiuxetan, arsenic trioxide, porfimer sodium, cetuximab, thioTEPA, altretamine, fulvestrant, exemestane, rituximab, alemtuzumab, dexamethasone, bicalutamide, chlorambucil, and valrubicin.

In other embodiments the present invention relates to a method of using a pharmaceutical composition comprising compound A or B and a pharmaceutically acceptable carrier comprising one or more additional therapeutic agents. The additional therapeutic agents may include, without limitation, an AKT inhibitor, alkylating agent, all-trans retinoic acid, antiandrogen, azacitidine, BCL2 inhibitor, BCL-XL inhibitor, BCR-ABL inhibitor, BTK inhibitor, BTK/LCK/LYN inhibitor, CDK1/2/4/6/7/9 inhibitor, CDK4/6 inhibitor, CDK9 inhibitor, CBP/p300 inhibitor, EGFR inhibitor, endothelin receptor antagonist, ERK inhibitor, farnesyltransferase inhibitor, FLT3 inhibitor, glucocorticoid receptor agonist, HDM2 inhibitor, histone deacetylase inhibitor, IKKβ inhibitor, immunomodulatory drug (IMiD), ingenol, ITK inhibitor, JAK1/JAK2/JAK3/TYK2 inhibitor, MEK inhibitor such as, but not limited to trametinib, selumetinib, and cobimetinib, midostaurin, MTOR inhibitor, PI3 kinase inhibitor, dual PI3 kinase/MTOR inhibitor, proteasome inhibitor, protein kinase C agonist, SUV39H1 inhibitor, TRAIL, VEGFR2 inhibitor, Wnt/β-catenin signaling inhibitor, decitabine, and anti-CD20 monoclonal antibody.

In other embodiments the present invention relates to a pharmaceutical composition comprising compound A or B and a pharmaceutically acceptable carrier comprising therapeutically effective amounts of one or more additional therapeutic agents, wherein said additional therapeutic agents are immune checkpoint inhibitors and are selected from the group consisting of CTLA4 inhibitors such as, but not limited to ipilimumab and tremelimumab; PD1 inhibitors such as, but not limited to pembrolizumab, and nivolumab; PDL1 inhibitors such as, but not limited to atezolizumab (formerly MPDL3280A), MEDI4736, avelumab, PDR001; 4 1BB or 4 1BB ligand inhibitors such as, but not limited to urelumab and PF-05082566; r OX40 ligand agonists such as, but not limited to MEDI6469; GITR inhibitors such as, but not limited to TRX518; CD27 inhibitors such as, but not limited to varlilumab; TNFRSF25 or TL1A inhibitors; CD40 agonists such as, but not limited to CP-870893; HVEM or LIGHT or LTA or BTLA or CD160 inhibitors; LAG3 inhibitors such as, but not limited to BMS-986016; TIM3 inhibitors; Siglecs inhibitors; ICOS or ICOS ligand agonists; B7 H3 inhibitors such as, but not limited to MGA271; B7 H4 inhibitors; VISTA inhibitors; HHLA2 or TMIGD2 inhibitors; inhibitors of Butyrophilins, including BTNL2 inhibitors; CD244 or CD48 inhibitors; inhibitors of TIGIT and PVR family members; KIRs inhibitors such as, but not limited to lirilumab; inhibitors of ILTs and LIRs; NKG2D and NKG2A inhibitors such as, but not limited to IPH2201; inhibitors of MICA and MICB; CD244 inhibitors; CSF1R inhibitors such as, but not limited to emactuzumab, cabiralizumab, pexidartinib, ARRY382, BLZ945; IDO inhibitors such as, but not limited to INCB024360; TGFβ inhibitors such as, but not limited to galunisertib; adenosine or CD39 or CD73 inhibitors; CXCR4 or CXCL12 inhibitors such as, but not limited to ulocuplumab and (3S,6S,9S,12R,17R,20S,23S,26S,29S,34aS)—N—((S)-1-amino-5-guanidino-1-oxopentan-2-yl)-26,29-bis(4-aminobutyl)-17-((S)-2-((S)-2-((S)-2-(4-fluorobenzamido)-5-guanidinopentanamido)-5-guanidinopentanamido)-3-(naphthalen-2-yl)propanamido)-6-(3-guanidinopropyl)-3,20-bis(4-hydroxybenzyl)-1,4,7,10,18,21,24,27,30-nonaoxo-9,23-bis(3-ureidopropyl)triacontahydro-1H,16H-pyrrolo[2,1-p][1,2]dithia[5,8,11,14,17,20,23,26,29]nonaazacyclodotriacontine-12-carboxamide BKT140; phosphatidylserine inhibitors such as, but not limited to bavituximab; SIRPA or CD47 inhibitors such as, but not limited to CC-90002; VEGF inhibitors such as, but not limited to bevacizumab; and neuropilin inhibitors such as, but not limited to MNRP1685A.

In using the pharmaceutical compositions of the compounds described herein, pharmaceutically acceptable carriers can be either solid or liquid. Solid forms include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa., which is hereby incorporated by reference in its entirety.

Liquid form preparations include solutions, suspensions and emulsions. For example, water or water-propylene glycol solutions for parenteral injection or addition of sweeteners and opacifiers for oral solutions, suspensions and emulsions. Liquid form preparations may also include solutions for intranasal administration.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Aerosol preparations suitable for inhalation may also be used. These preparations may include solutions and solids in powder form, which may be in combination with a pharmaceutically acceptable carrier, such as an inert compressed gas, e.g., nitrogen.

Also contemplated for use are solid form preparations that are intended to be converted, shortly before use, to liquid form preparations for either oral or parenteral administration. Such liquid forms include solutions, suspensions and emulsions.

### Combination Therapies

As previously noted, the compounds described herein can be used alone or in combination with other agents. For example, the compounds can be administered together with a cancer targeted therapeutic agent, cancer-targeted biological, immune checkpoint inhibitor, or a chemotherapeutic agent. In another embodiment compound A or B can be used alone or singularly. The agent can be administered together with or sequentially with a compound described herein in a combination therapy.

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X—Y—X, X—X—Y, Y—X—Y, Y—Y—X, X—X—Y—Y, etc.

In one embodiment, compound A or B is administered to a patient in need of treatment in combination of a therapeutic agent selected from cytotoxic agent, cisplatin, doxorubicin, etoposide, irinotecan, topotecan, paclitaxel, docetaxel, the epothilones, tamoxifen, 5-fluorouracil, methotrexate, temozolomide, cyclophosphamide, lonafarib, tipifarnib, 4-((5-((4-(3-chlorophenyl)-3-oxopiperazin-1-yl)methyl)-1H-imidazol-1-yl)methyl)benzonitrile hydrochloride, (R)-1-((1H-imidazol-5-yl)methyl)-3-benzyl-4-(thiophen-2-ylsulfonyl)-2,3,4,5-tetrahydro-1H-benzo diazepine-7-carbonitrile, cetuximab, imatinib, interferon alfa-2b, pegylated interferon alfa-2b, aromatase combinations, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, leucovorin, oxaliplatin, pentostatine, vinblastine, vincristine, vindesine, bleomycin, dactinomycin, daunorubicin, epirubicin, idarubicin, mithramycin, deoxycoformycin, mitomycin-C, L-asparaginase, teniposide 17α-ethinyl estradiol, diethylstilbestrol, testosterone, prednisone, fluoxymesterone, dromostanolone propionate, testolactone, megestrol acetate, methylprednisolone, methyltestosterone, prednisolone, triamcinolone, chlorotrianisene, 17α-hydroxyprogesterone, aminoglutethimide, estramustine, medroxyprogesterone acetate, leuprolide acetate, flutamide, toremifene citrate, goserelin acetate, carboplatin, hydroxyurea, amsacrine, procarbazine, mitotane, mitoxantrone, levamisole, vinorelbine, anastrazole, letrozole, capecitabine, raloxifene, droloxafine, hexamethylmelamine, bevacizumab, trastuzumab, tositumomab, bortezomib, ibritumomab tiuxetan, arsenic trioxide, porfimer sodium, cetuximab, thioTEPA, altretamine, fulvestrant, exemestane, rituximab, alemtuzumab, dexamethasone, bicalutamide, chlorambucil, and valrubicin.

In one embodiment, compound A or B is administered to a patient in need of treatment in combination with an immune checkpoint inhibitors selected from CTLA4 inhibitors such as, but not limited to ipilimumab and tremelimumab; PD1 inhibitors such as, but not limited to pembrolizumab, and nivolumab; PDL1 inhibitors such as, but not limited to atezolizumab (formerly MPDL3280A), MEDI4736, avelumab, PDR001; 4 1BB or 4 1BB ligand inhibitors such as, but not limited to urelumab and PF-05082566; OX40 ligand agonists such as, but not limited to MEDI6469; GITR inhibitors such as, but not limited to TRX518; CD27 inhibitors such as, but not limited to varlilumab; TNFRSF25 or TL1A inhibitors; CD40 ligand agonists such as, but not limited to CP-870893; HVEM or LIGHT or LTA or BTLA or CD160 inhibitors; LAG3 inhibitors such as, but not limited to BMS-986016; TIM3 inhibitors; Siglecs inhibitors; ICOS or ICOS ligand inhibitors; B7 H3 inhibitors such as, but not limited to MGA271; B7 H4 inhibitors; VISTA inhibitors; HHLA2 or TMIGD2 inhibitors; inhibitors of Butyrophilins, including BTNL2 inhibitors; CD244 or CD48 inhibitors; inhibitors of TIGIT and PVR family members; KIRs inhibitors such as, but not limited to lirilumab; inhibitors of ILTs and LIRs; NKG2D and NKG2A inhibitors such as, but not limited to IPH2201; inhibitors of MICA and MICB; CD244 inhibitors; CSF1R inhibitors such as, but not limited to emactuzumab, cabiralizumab, pexidartinib, ARRY382, and BLZ945; IDO inhibitors such as, but not limited to INCB024360; TGFβ inhibitors such as, but not limited to galunisertib; adenosine or CD39 or CD73 inhibitors; CXCR4 or CXCL12 inhibitors such as, but not limited to ulocuplumab and (3S,6S,9S,12R,17R,20S,23S,26S,29S,34aS)—N—((S)-1-amino-5-guanidino-1-oxopentan-2-yl)-26,29-bis(4-aminobutyl)-17-((S)-2-((S)-2-((S)-2-(4-fluorobenzamido)-5-guanidinopentanamido)-5-guanidinopentanamido)-3-(naphthalen-2-yl)propanamido)-6-(3-guanidinopropyl)-3,20-bis(4-hydroxybenzyl)-1,4,7,10,18,21,24,27,30-nonaoxo-9,23-bis(3-ureidopropyl)triacontahydro-1H,16H-pyrrolo[2,1-p][1,2]dithia[5,8,11,14,17,20,23,26,29]nonaazacyclodotriacontine-12-carboxamide BKT140; phosphatidylserine inhibitors such as, but not limited to bavituximab; SIRPA or CD47 inhibitors such as, but not limited to CC-90002; VEGF inhibitors such as, but not limited to bevacizumab; or neuropilin inhibitors such as, but not limited to MNRP1685A.

According to another embodiment of the invention, additional therapeutic agents may be used in combination with Compound A or B. These agents include, without limitation, an AKT inhibitor, alkylating agent, all-trans retinoic acid, antiandrogen, azacitidine, BCL2 inhibitor, BCL-XL inhibitor, BCR-ABL inhibitor, BTK inhibitor, BTK/LCK/LYN inhibitor, CDK1/2/4/6/7/9 inhibitor, CDK4/6 inhibitor, CDK9 inhibitor, CBP/p300 inhibitor, EGFR inhibitor, endothelin receptor antagonist, ERK inhibitor, farnesyltransferase inhibitor, FLT3 inhibitor, glucocorticoid receptor agonist, HDM2 inhibitor, histone deacetylase inhibitor, IKKβ inhibitor, immunomodulatory drug (IMiD), ingenol, ionizing radiation, ITK inhibitor, JAK1/JAK2/JAK3/TYK2 inhibitor, MEK inhibitor such as, but not limited to trametinib, selumetinib, and cobimetinib, midostaurin, MTOR inhibitor, PI3 kinase inhibitor, dual PI3 kinase/MTOR inhibitor, proteasome inhibitor, protein kinase C agonist, SUV39H1 inhibitor, TRAIL, VEGFR2 inhibitor, Wnt/β-catenin signaling inhibitor, decitabine, and anti-CD20 monoclonal antibody.

### Dosage

In some embodiments where a compound A or B is used in combination with an other agent for a treatment protocol, the composition may be administered together or in a “dual-regimen” wherein the two therapeutics are dosed and administered separately. When the compound A or B and the additional agent are dosed separately, the typical dosage administered to the subject in need of the treatment is typically from about 5 mg per day and about 5000 mg per day and, in other embodiments, from about 50 mg per day and about 1000 mg per day. Other dosages may be from about 10 mmol up to about 250 mmol per day, from about 20 mmol to about 70 mmol per day or even from about 30 mmol to about 60 mmol per day.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. A typical recommended daily dosage regimen for oral administration can range from about 1 mg/day to about 500 mg/day or 1 mg/day to 200 mg/day, in a single dose, or in two to four divided doses. In one embodiment, the typical daily dose regimen is 150 mg.

Compounds of the present disclosure with or without the additional agent described herein may be administered by any suitable route. The compound can be administrated orally (e.g., dietary) in capsules, suspensions, tablets, pills, dragees, liquids, gels, syrups, slurries, and the like. Methods for encapsulating compositions (such as in a coating of hard gelatin or cyclodextran) are known in the art (Baker, et al., “Controlled Release of Biological Active Agents”, John Wiley and Sons, 1986, which is hereby incorporated by reference in its entirety). The compounds can be administered to the subject in conjunction with an acceptable pharmaceutical carrier as part of a pharmaceutical composition. The formulation of the pharmaceutical composition will vary according to the route of administration selected. Suitable pharmaceutical carriers may contain inert ingredients which do not interact with the compound. The carriers are biocompatible, i.e., non-toxic, non-inflammatory, non-immunogenic and devoid of other undesired reactions at the administration site.

Illustrative pharmaceutical compositions are tablets and gelatin capsules comprising a Compound of the Invention and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

If formulated as a fixed dose, such combination products employ the compounds of this invention within the dosage range described herein, or as known to those skilled in the art.

Since the compounds of this invention (Compounds A and B) are intended for use in pharmaceutical compositions a skilled artisan will understand that they can be provided in substantially pure forms for example, at least 60% pure, at least 75% pure, at least 85% pure, and at least 98% pure (w/w). The pharmaceutical preparation may be in a unit dosage form. In such form, the preparation is subdivided into suitably sized unit doses containing appropriate quantities of compounds A or B, e.g., an effective amount to achieve the desired purpose as described herein.

Section 1—Important Structural Comparisons vs. Biological Activity with WO/2008/034008 and WO/2013/184119

WO/2008/034008 describes various kinases that cause or contribute to the pathogenesis of various proliferative diseases, said kinases including BRaf, CRaf, Abl, KDR(VEGFR2), EGFR/HER1, HER2, HER3, c-MET, FLT-3, PDGFR-α, PDGFR-β, p38, c-KIT, JAK2 family. The disclosure of this PCT application explicitly demonstrates selective inhibition toward Braf and CRaf kinases using analogues of Compounds A and B described herein. Concomitantly, WO/2013/184119 describes the inhibition of mutant c-KIT with Compounds A and B. However, WO/2013/184119 also discloses that c-KIT and PDGFRα mutations are mutually exclusive in GIST. This is because most GISTs have primary activating mutations in the genes encoding the closely related RTKs c-KIT (75-80% of GIST) or PDGFRα (8% of the non-c-KIT mutated GIST) in a mutually exclusive manner.

In the present application, the inexorable mutual exclusivity between c-KIT and PDGFRα mutations in GIST patients is reconciled with the finding that Compounds A and B can treat both patient populations. In fact, it has unexpectedly been found that compounds A and B which are known to inhibit c-KIT mutant also inhibit wild-type and oncogenic mutated PDGFR kinases, oncogenic fusion protein forms of PDGFRα kinase, and PDGFRα amplified cancers contrary to the prior disclosures of WO/2008/034008 and WO/2013/184119. The experimental data described below further corroborate this discovery. A direct application of this finding is the treatment of cancer patient sub-populations that express resistant forms of cancers described herein and that are PDGFR-derived.

### EXAMPLES

### Biological Data

It has been found that compounds A and B unexpectedly inhibit wild-type and oncogenic mutated PDGFR kinases, oncogenic fusion protein forms of PDGFRα kinase, and PDGFRα mutated or amplified cancers. Characterization of this unexpected finding was undertaken in biochemical assays, cellular assays, and in in vivo clinical evaluation in cancer patients.

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1. Inhibition of Wild Type PDGFRα Enzyme Activity

### Biochemical Assay for PDGFRα (GenBank Accession Number: NP_006197)

The activity of PDGFRα kinase was determined spectroscopically using a coupled pyruvate kinase/lactate dehydrogenase assay that continuously monitors the ATP hydrolysis-dependent oxidation of NADH (e.g., Schindler et al. Science (2000) 289: 1938-1942, which is hereby incorporated by reference in its entirety). Assays were conducted in 384-well plates (100 μL final volume) using 4.8 nM PDGFRA (DeCode Biostructures, Bainbridge Island, Wash.), 5 units pyruvate kinase, 7 units lactate dehydrogenase, 1 mM phosphoenol pyruvate, 0.28 mM NADH, 2.5 mg/mL PolyEY and 0.5 mM ATP in assay buffer (90 mM Tris, pH 7.5, 18 mM MgCl₂, 1 mM DTT, and 0.2% octyl-glucoside). Inhibition of PDGFRA was measured after adding serial diluted test compound (final assay concentration of 1% DMSO). A decrease in absorption at 340 nm was monitored continuously for 6 hours at 30° C. on a multi-mode microplate reader (BioTek, Winooski, Vt.). The reaction rate was calculated using the 1-2 h time frame. The reaction rate at each concentration of compound was converted to percent inhibition using controls (i.e. reaction with no test compound and reaction with a known inhibitor) and IC₅₀ values were calculated by fitting a four-parameter sigmoidal curve to the data using Prism (GraphPad, San Diego, Calif.).

PDGFRα protein sequence (residues 550-1089 with a
N-terminal GST-tag; Genbank Seq. ID No.: 1)
MEHHHHHHHHMAPILGYWKIKGLVQPTRLLLEYLEEKYEEHLYERDEGD
KWRNKKFELGLEFPNLPYYIDGDVKLTQSMAIIRYIADKHNMLGGCPKE
RAEISMLEGAVLDIRYGVSRIAYSKDFETLKVDFLSKLPEMLKMFEDRL
CHKTYLNGDHVTHPDFMLYDALDVVLYMDPMCLDAFPKLVCFKKRIEAI
PQIDKYLKSSKYIAWPLQGWQATFGGGDHPPKSDLVPRHNQTSLYKKAG
FEGDRTMKQKPRYEIRWRVIESISPDGHEYIYVDPMQLPYDSRWEFPRD
GLVLGRVLGSGAFGKVVEGTAYGLSRSQPVMKVAVKMLKPTARSSEKQA
LMSELKIMTHLGPHLNIVNLLGACTKSGPIYIITEYCFYGDLVNYLHKN
RDSFLSHHPEKPKKELDIFGLNPADESTRSYVILSFENNGDYMDMKQAD
TTQYVPMLERKEVSKYSDIQRSLYDRPASYKKKSMLDSEVKNLLSDDNS
EGLTLLDLLSFTYQVARGMEFLASKNCVHRDLAARNVLLAQGKIVKICD
FGLARDIMHDSNYVSKGSTFLPVKWMAPESIFDNLYTTLSDVWSYGILL
WEIFSLGGTPYPGMMVDSTFYNKIKSGYRMAKPDHATSEVYEIMVKCWN
SEPEKRPSFYHLSEIVENLLPGQYKKSYEKIHLDFLKSDHPAVARMRVD
SDNAYIGVTYKNEEDKLKDWEGGLDEQRLSADSGYIIPLPDIDPVPEEE
DLGKRNRHSSQTSEESAIETGSSSSTFIKREDETIEDIDMMDDIGIDSS
DLVEDSFL

Compound A inhibited recombinant wild type PDGFRα enzyme activity with an IC₅₀ value of 12 nM. Compound B inhibited recombinant wild type PDGFRα enzyme activity with an IC₅₀ value of 6 nM.

### Example 2. Inhibition of D842V Mutant PDGFRα Enzyme Activity

### Biochemical Assay for PDGFRα D842V (GenBank Accession Number: NP_006197)

The activity of PDGFRA D842V kinase was determined spectroscopically using a coupled pyruvate kinase/lactate dehydrogenase assay that continuously monitors the ATP hydrolysis-dependent oxidation of NADH (e.g., Schindler et al. Science (2000) 289: 1938-1942, which is hereby incorporated by reference in its entirety). Assays were conducted in 384-well plates (100 μL final volume) using 3 nM PDGFRA D842V (Invitrogen, Carlsbad, Calif.), 5 units pyruvate kinase, 7 units lactate dehydrogenase, 1 mM phosphoenol pyruvate, 0.28 mM NADH, 2.5 mg/mL PolyEY and 0.5 mM ATP in assay buffer (90 mM Tris, pH 7.5, 18 mM MgCl₂, 1 mM DTT, and 0.2% octyl-glucoside). Inhibition of PDGFRA D842V was measured after adding serial diluted test compound (final assay concentration of 1% DMSO). A decrease in absorption at 340 nm was monitored continuously for 6 hours at 30° C. on a multi-mode microplate reader (BioTek, Winooski, Vt.). The reaction rate was calculated using the 2-3 h time frame. The reaction rate at each concentration of compound was converted to percent inhibition using controls (i.e. reaction with no test compound and reaction with a known inhibitor) and IC₅₀ values were calculated by fitting a four-parameter sigmoidal curve to the data using Prism (GraphPad, San Diego, Calif.).

PDGFRα D842V protein sequence (residues 550-1089
with a N-terminal HIS-GST-tag; Genbank Seq. ID
No.: 2)
MAPILGYWKIKGLVQPTRLLLEYLEEKYEEHLYERDEGDKWRNKKFELG
LEFPNLPYYIDGDVKLTQSMAIIRYIADKHNMLGGCPKERAEISMLEGA
VLDIRYGVSRIAYSKDFETLKVDFLSKLPEMLKMFEDRLCHKTYLNGDH
VTHPDFMLYDALDVVLYMDPMCLDAFPKLVCFKKRIEAIPQIDKYLKSS
KYIAWPLQGWQATFGGGDHPPKSDLVPRHNQTSLYKKAGFEGDRTMKQK
PRYEIRWRVIESISPDGHEYIYVDPMQLPYDSRWEFPRDGLVLGRVLGS
GAFGKVVEGTAYGLSRSQPVMKVAVKMLKPTARSSEKQALMSELKIMTH
LGPHLNIVNLLGACTKSGPIYIITEYCFYGDLVNYLHKNRDSFLSHHPE
KPKKELDIFGLNPADESTRSYVILSFENNGDYMDMKQADTTQYVPMLER
KEVSKYSDIQRSLYDRPASYKKKSMLDSEVKNLLSDDNSEGLTLLDLLS
FTYQVARGMEFLASKNCVHRDLAARNVLLAQGKIVKICDFGLARVIMHD
SNYVSKGSTFLPVKWMAPESIFDNLYTTLSDVWSYGILLWEIFSLGGTP
YPGMMVDSTFYNKIKSGYRMAKPDHATSEVYEIMVKCWNSEPEKRPSFY
HLSEIVENLLPGQYKKSYEKIHLDFLKSDHPAVARMRVDSDNAYIGVTY
KNEEDKLKDWEGGLDEQRLSADSGYIIPLPDIDPVPEEEDLGKRNRHSS
QTSEESAIETGSSSSTFIKREDETIEDIDMMDDIGIDSSDLVEDSFL

Compound A inhibited recombinant D842V mutant PDGFRα enzyme activity with an IC₅₀ value of 42 nM. Compound B inhibited recombinant D842V mutant PDGFRα enzyme activity with an IC₅₀ value of 20 nM.

### Example 3. Inhibition of Wild Type PDGFRβ Enzyme Activity

### Biochemical Assay for PDGFRB (GenBank Accession Number: NP_002600)

The activity of PDGFRβ kinase was determined spectroscopically using a coupled pyruvate kinase/lactate dehydrogenase assay that continuously monitors the ATP hydrolysis-dependent oxidation of NADH (e.g., Schindler et al. Science (2000) 289: 1938-1942, which is hereby incorporated by reference in its entirety). Assays were conducted in 384-well plates (100 μL final volume) using 9 nM PDGFRB (DeCode Biostructures, Bainbridge Island, Wash.), 5 units pyruvate kinase, 7 units lactate dehydrogenase, 1 mM phosphoenol pyruvate, 0.28 mM NADH, 2.5 mg/mL PolyEY and 0.5 mM ATP in assay buffer (90 mM Tris, pH 7.5, 18 mM MgCl₂, 1 mM DTT, and 0.2% octyl-glucoside). Inhibition of PDGFRB was measured after adding serial diluted test compound (final assay concentration of 1% DMSO). A decrease in absorption at 340 nm was monitored continuously for 6 hours at 30° C. on a multi-mode microplate reader (BioTek, Winooski, Vt.). The reaction rate was calculated using the 2-3 h time frame. The reaction rate at each concentration of compound was converted to percent inhibition using controls (i.e. reaction with no test compound and reaction with a known inhibitor) and IC₅₀ values were calculated by fitting a four-parameter sigmoidal curve to the data using Prism (GraphPad, San Diego, Calif.).

PDGFRβ protein sequence (residues 557-1106 with a
N-terminal HIS-GST-tag; Genbank Seq. ID No.: 3)
MEHHHHHHHHMAPILGYWKIKGLVQPTRLLLEYLEEKYEEHLYERDEGD
KWRNKKFELGLEFPNLPYYIDGDVKLTQSMAIIRYIADKHNMLGGCPKE
RAEISMLEGAVLDIRYGVSRIAYSKDFETLKVDFLSKLPEMLKMFEDRL
CHKTYLNGDHVTHPDFMLYDALDVVLYMDPMCLDAFPKLVCFKKRIEAI
PQIDKYLKSSKYIAWPLQGWQATFGGGDHPPKSDLVPRHNQTSLYKKAG
FEGDRTMQKKPRYEIRWKVIESVSSDGHEYIYVDPMQLPYDSTWELPRD
QLVLGRTLGSGAFGQVVEATAHGLSHSQATMKVAVKMLKSTARSSEKQA
LMSELKIMSHLGPHLNVVNLLGACTKGGPIYIITEYCRYGDLVDYLHRN
KHTFLQHHSDKRRPPSAELYSNALPVGLPLPSHVSLTGESDGGYMDMSK
DESVDYVPMLDMKGDVKYADIESSNYMAPYDNYVPSAPERTCRATLINE
SPVLSYMDLVGFSYQVANGMEFLASKNCVHRDLAARNVLICEGKLVKIC
DFGLARDIMRDSNYISKGSTFLPLKWMAPESIFNSLYTTLSDVWSFGIL
LWEIFTLGGTPYPELPMNEQFYNAIKRGYRMAQPAHASDEIYEIMQKCW
EEKFEIRPPFSQLVLLLERLLGEGYKKKYQQVDEEFLRSDHPAILRSQA
RLPGFHGLRSPLDTSSVLYTAVQPNEGDKDYIIPLPDPKPEVADEGPLE
GSPSLASSTLNEVNTSSTISCDSPLEPQDEPEPEPQLELQVEPEPELEQ
LPDSGCPAPRAEAEDSFL

Compound A inhibited recombinant wild type PDGFRβ enzyme activity with an IC₅₀ value of 9 nM. Compound B inhibited recombinant wild type PDGFRβ enzyme activity with an IC₅₀ value of 5 nM.

### Example 4. Proliferation Inhibition of D842V Mutant PDGFRα Expressed in Ba/F3 Cells

### BaF3 PDGFRα D842V Cell Culture

BaF3 cells were transfected with a construct encoding D842V PDGFRα and selected for IL-3 independence. Briefly, cells were grown in RPMI 1640 media supplemented with 10% characterized fetal bovine serum (Invitrogen, Carlsbad, Calif.), 1 unit/mL penicillin G, 1 μg/ml streptomycin, and 0.29 mg/mL L-glutamine at 37 degrees Celsius, 5% CO2, 95% humidity.

### BaF3 PDGFRα D842V Cell Proliferation Assays

A serial dilution of test compound was dispensed into a 96-well black clear bottom plate (Corning, Corning, N.Y.). Ten thousand cells were added per well in 200 μL complete growth medium. Plates were incubated for 67 hours at 37 degrees Celsius, 5% CO₂, 95% humidity. At the end of the incubation period 40 μL of a 440 μM solution of resazurin (Sigma, St. Louis, Mo.) in PBS was added to each well and plates were incubated for an additional 5 hours at 37 degrees Celsius, 5% CO₂, 95% humidity. Plates were read on a Synergy2 reader (Biotek, Winooski, Vt.) using an excitation of 540 nm and an emission of 600 nm. Data was analyzed using Prism software (GraphPad, San Diego, Calif.) to calculate IC₅₀ values.

Compound A inhibited proliferation of D842V mutant PDGFRα BaF3 cells with an IC₅₀ value of 36 nM. Compound B inhibited proliferation of D842V mutant PDGFRα BaF3 cells with an IC₅₀ value of 42 nM.

### Example 5. Phosphorylation Inhibition of D842V Mutant PDGFRα Expressed in BaF3 Cells

### BaF3 PDGFRα D842V Cell Culture

BaF3 cells were transfected with a construct encoding D842V PDGFRα and selected for IL-3 independence. Briefly, cells were grown in RPMI 1640 media supplemented with 10% characterized fetal bovine serum (Invitrogen, Carlsbad, Calif.), 1 unit/mL penicillin G, 1 μg/ml streptomycin, and 0.29 mg/mL L-glutamine at 37 degrees Celsius, 5% CO2, 95% humidity.

### BaF3 PDGFRα D842V Western Blots

Two million cells per well suspended in serum-free RPMI 1640 were added to a 24-well tissue-culture treated plate. A serial dilution of test compound was added to plates containing cells and plates were incubated for 4 hours at 37 degrees Celsius, 5% CO₂, 95% humidity. Cells were washed with PBS, then lysed. Cell lysates were separated by SDS-PAGE and transferred to PVDF. Phospho-PDGFRα (Tyr754) was detected using an antibody from Cell Signaling Technology (Beverly, Mass.), ECL Plus detection reagent (GE Healthcare, Piscataway, N.J.) and a Molecular Devices Storm 840 phosphorimager in fluorescence mode. Blots were stripped and probed for total PDGFRα using an antibody from Cell Signaling Technology (Beverly, Mass.). IC50 values were calculated using Prism software (GraphPad, San Diego, Calif.).

Compound A inhibited phosphorylation of D842V mutant PDGFRα expressed in BaF3 cells with an IC₅₀ value of 24 nM. Compound B inhibited phosphorylation of D842V mutant PDGFRα expressed in BaF3 cells with an IC₅₀ value of 26 nM.

### Example 6. Phosphorylation Inhibition of V561D Mutant PDGFRα Expressed in CHO Cells

Chinese hamster ovary (CHO) cells were transiently transfected with mutated V561D PDGFRA cDNA construct cloned into the pcDNA3.1 plasmid (Invitrogen, Carlsbad, Calif.). Twenty-four hours post transfection, cells were treated with various concentrations of compound for 90 minutes. Protein lysates from cells were prepared and subjected to immunoprecipitation using anti-PDGFRA antibody (SC-20, Santa Cruz Biotechnology, Santa Cruz, Calif.), followed by sequential immunoblotting for phosphotyrosine using a monoclonal antibody (PY-20, BD Transduction Labs, Sparks, Md.) or total PDGFRα (SC-20, Santa Cruz Biotechnology, Santa Cruz, Calif.). Densitometry was performed to quantify drug effect using Photoshop 5.1 software, with the level of phospho-PDGFRα normalized to total protein. Densitometry experimental results were analyzed using Calcusyn 2.1 software (Biosoft, Cambridge, UK) to mathematically determine the IC₅₀ values.

Compound A inhibited phosphorylation of V561D mutant PDGFR□ expressed in CHO cells with an IC₅₀ value of 25 nM.

### Example 7. Phosphorylation Inhibition of Exon 18 842-845 Deletion Mutant PDGFRα Expressed in CHO Cells

Chinese hamster ovary (CHO) cells were transiently transfected with mutated ΔD842-H845 PDGFRA cDNA construct cloned into the pcDNA3.1 plasmid (Invitrogen, Carlsbad, Calif.). Twenty-four hours post transfection, cells were treated with various concentrations of compound for 90 minutes. Protein lysates from cells were prepared and subjected to immunoprecipitation using anti-PDGFRA antibody (SC-20, Santa Cruz Biotechnology, Santa Cruz, Calif.), followed by sequential immunoblotting for phosphotyrosine using a monoclonal antibody (PY-20, BD Transduction Labs, Sparks, Md.) or total PDGFRα (SC-20, Santa Cruz Biotechnology, Santa Cruz, Calif.). Densitometry was performed to quantify drug effect using Photoshop 5.1 software, with the level of phospho-PDGFRA normalized to total protein. Densitometry experimental results were analyzed using Calcusyn 2.1 software (Biosoft, Cambridge, UK) to mathematically determine the IC₅₀ values.

Compound A inhibited phosphorylation of exon 18 842-845 deletion mutant PDGFRα expressed in CHO cells with an IC₅₀ value of 77 nM.

### Example 8. Proliferation Inhibition of FIP1L1-PDGFRα Fusion in EOL-1 Cells

### EOL-1 (FIPIL1/PDGFRα Fusion) Cell Culture

EOL-1 cells were grown in RPMI 1640 media supplemented with 10% characterized fetal bovine serum (Invitrogen, Carlsbad, Calif.), 1 unit/mL penicillin G, 1 μg/ml streptomycin, and 0.29 mg/mL L-glutamine at 37 degrees Celsius, 5% CO2, 95% humidity.

### EOL-1 Cell Proliferation Assays

A serial dilution of test compound was dispensed into a 96-well black clear bottom plate (Corning, Corning, N.Y.). Ten thousand cells were added per well in 200 μL complete growth medium. Plates were incubated for 67 hours at 37 degrees Celsius, 5% CO2, 95% humidity. At the end of the incubation period 40 μL of a 440 μM solution of resazurin (Sigma, St. Louis, Mo.) in PBS was added to each well and plates were incubated for an additional 5 hours at 37 degrees Celsius, 5% CO2, 95% humidity. Plates were read on a Synergy2 reader (Biotek, Winooski, Vt.) using an excitation of 540 nm and an emission of 600 nm. Data was analyzed using Prism software (GraphPad, San Diego, Calif.) to calculate IC50 values.

Compound A inhibited proliferation of FIP1L1-PDGFRα fusion in EOL-1 cells with an IC₅₀ value of 0.029 nM. Compound B inhibited proliferation of FIP1L1-PDGFRα fusion in EOL-1 cells with an IC₅₀ value of 0.018 nM.

### Example 9. Phosphorylation Inhibition of FIP1L1-PDGFRα Fusion in EOL-1 Cells

### EOL-1 (FIPIL1/PDGFRα Fusion) Cell Culture

EOL-1 cells were grown in RPMI 1640 media supplemented with 10% characterized fetal bovine serum (Invitrogen, Carlsbad, Calif.), 1 unit/mL penicillin G, 1 μg/ml streptomycin, and 0.29 mg/mL L-glutamine at 37 degrees Celsius, 5% CO2, 95% humidity.

### EOL-1 Western Blots

Two million cells per well suspended in serum-free RPMI 1640 were added to a 24-well tissue-culture treated plate. A serial dilution of test compound was added to plates containing cells and plates were incubated for 4 hours at 37 degrees Celsius, 5% CO2, 95% humidity. Cells were washed with PBS, then lysed. Cell lysates were separated by SDS-PAGE and transferred to PVDF. Phospho-PDGFRα (Tyr754) was detected using an antibody from Cell Signaling Technology (Beverly, Mass.), ECL Plus detection reagent (GE Healthcare, Piscataway, N.J.) and a Molecular Devices Storm 840 phosphorimager in fluorescence mode. Blots were stripped and probed for total PDGFRα using an antibody from Cell Signaling Technology (Beverly, Mass.). IC50 values were calculated using Prism software (GraphPad, San Diego, Calif.).

Compound A inhibited phosphorylation of FIP1L1-PDGFRα fusion in EOL-1 cells with an IC₅₀ value of 0.12 nM. Compound B inhibited phosphorylation of FIP1L1-PDGFRα fusion in EOL-1 cells with an IC₅₀ value of <0.1 nM.

### Example 10. Treatment of Human Cancer Patients with PDGFRα D842V Mutation

The clinical study protocol DCC-2618-01-001 “A Multicenter Phase 1, Open-Label Study of Compound A to Assess Safety, Tolerability, and Pharmacokinetics in Patients with Advanced Malignancies” is the first-in-human study of Compound A (ClinicalTrials.gov Identifier: NCT02571036). The objectives of this dose-escalation study are to evaluate the safety, tolerability, pharmacokinetics (PK), pharmacodynamics (PD) and preliminary antitumor activity of Compound A. The study medication is administered orally either once or twice daily at escalating doses within the range from 20 mg BID to 200 mg BID. Preliminary antitumor activity was measured by CT scans according to RECIST 1.1 every other cycle (every 56 days). Pharmacodynamics effects were measured as a reduction in mutation allele frequency (MAF) in plasma cell-free (cf) DNA and analyzed with Guardant 360 v2.9 or v2.10 (Guardant Health, Redwood City, Calif.), a. 73-gene next generation sequencing panel.

All patients had to have progressive disease on standard of care treatment and would rapidly progress without treatment. Three patients with PDGFRα-mutated Gastrointestinal Stromal Tumors (GIST) were enrolled in the study. The PDGFRα D842V mutation was identified in each patient by tumor biopsy. Based on non-clinical data and the available pharmacokinetic data from study DCC-2618-01-001, dose levels of >50 mg BID (daily dose equivalent 100 mg) were sufficient to lead to tumor control i.e. growth arrest in these advanced sarcomas of PDGFRα D842V mutation-dependent tumors in patients suffering from GIST. Out of 3 evaluable patients, 2 were enrolled at or above target-effective dose levels (150 mg QD and 100 mg BID). The other patient was enrolled at 30 mg BID and progressed after 2 treatment cycles of 28 days. The patient at 100 mg BID is now in Cycle 11 (>40 weeks) and continues to benefit from treatment. The most recent tumor assessment confirmed ‘Stable Disease’ according to RECIST 1.1. Tumor assessments throughout the study revealed some tumor reduction (5 to 10%) including the most recent one after Cycle 9 (36 weeks). The patient treated at the 150 mg QD dose level is now in Cycle 6 (>20 weeks) with stable disease per RECIST and has some tumor reduction observed. The 2 patients had 1 and 3 prior treatments with Tyrosine Kinase Inhibitors, respectively.

To date, cfDNA follow up data for PDGFRα D842V mutation allele frequency in plasma are available for the patient at 100 mg BID only. The PDGFRα D842V mutation was not detected by cfDNA at baseline, but at Cycle 3 Day 1 (8 weeks) post-treatment a frequency of 0.59% was detected. While the lack of D842V mutation detection at baseline might limit the ability to interpret the data, the fact that the mutation found in tumor tissue is “undetectable” i.e. below the limit of detection at 2 sequential analyses points (Cycle 5 Day 1 (16 weeks) and Cycle 7 Day 1 (24 weeks)) strongly supports the suppression of this PDGFRα D842V mutation due to treatment of human cancer patients with Compound A.

### Example 11. Treatment of a Human Glioblastoma Patient with PDGFRα Amplification

The clinical study protocol DCC-2618-01-001 “A Multicenter Phase 1, Open-Label Study of Compound A to Assess Safety, Tolerability, and Pharmacokinetics in Patients with Advanced Malignancies” is the first-in-human study of Compound A (ClinicalTrials.gov Identifier: NCT02571036). The objectives of this dose-escalation study are to evaluate the safety, tolerability, pharmacokinetics (PK), pharmacodynamics (PD) and preliminary antitumor activity of Compound A. The study medication is administered orally either once or twice daily at escalating doses within the range from 20 mg BID to 200 mg BID. Preliminary antitumor activity was measured by CT scans according to RANO (Revised Assessment in Neuro-Oncology) criteria every other cycle followed by after every 3^{rd} cycle (every 56 or 84 days). Pharmacodynamic effects were measured as a reduction in circulating tumor cells (CTC). Whole blood was enriched for CTCs in an OncoQuick tube. The CTC layer was incubated with an adenovirus that replicates and expresses GFP in cells with high levels of telomerase (Oncolys BioPharma Inc.). Cells were then incubated with fluorescently-labeled antibodies, fixed, and stained with DAPI. Cells positive for DAPI, GFP, PDGFRα and GFAP fluorescence were counted as circulating glioblastoma tumor cells using a BioTek Cytation 5 imager. Glial fibrillary acidic protein (GFAP) is unambiguously attributed to glial cells.

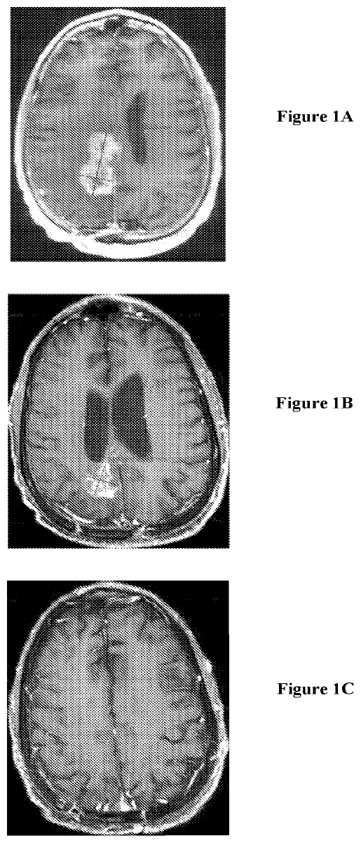

All patients had to have progressive disease on standard of care treatment and would rapidly progress without treatment. One patient with PDGFRα amplified glioblastoma (GBM; 6× amplified, 12 copies) was enrolled in the study at the 20 mg BID dose level. The patient had been treated initially with combined radio-chemotherapy followed by temozolomide alone and progressed after 3 months. The GBM patient is now in cycle 19 (>17 months on study) and continues to benefit from treatment. Since the tumor assessment after Cycle 12 (48 weeks), the patient has a ‘Partial Response’ according to the RANO criteria. FIG. 1A shows the MRI scan at baseline and FIG. 1C shows an MRI scan after cycle 12. FIG. 1B provided an additional proof of the tumor reduction after cycle 9.

The relevance of PDGFRα amplification has been assessed in pediatric and adult high-grade astrocytomas (HGA) including glioblastomas. A large study on primary human tissue suggests a significant prevalence of PDGFRα amplified HGA and indicates that PDGFRα amplification increases with grade and is associated with a less favorable prognosis in IDH1 mutant de novo GBMs (Philips et al., Brain Pathol. (2013) 23(5):565-73, which is hereby incorporated by reference in its entirety). Dunn et al., provide additional evidence that PDGFRα amplification is a driver genomic alteration for GBM (Dunn et al., Genes Dev. (2012) 26(8):756-84). Based on these findings, the pharmacodynamic effect, measured as a reduction in CTC observed in the GBM patient following treatment with Compound A, strongly supports that the partial response observed in the GBM patient is a result of treatment of a PDGFRα amplified tumor with Compound A. Double positive CTCs (PDGFRα+/GFAP+) were first measured at cycle 7 (28 weeks) with a frequency of 2.22 CTCs/mL. The frequency dropped in cycles 13 (52 weeks) and 17 (68 weeks) to 1.11 and 0.58 CTCs/mL, respectively.

### Example 12 Compound B is Formed Biosynthetically after Oral Administration of Compound A

The clinical study protocol DCC-2618-01-001 “A Multicenter Phase 1, Open-Label Study of Compound A to Assess Safety, Tolerability, and Pharmacokinetics in Patients with Advanced Malignancies” is the first-in-human study of Compound A (ClinicalTrials.gov Identifier: NCT02571036). The objectives of this dose-escalation study are to evaluate the safety, tolerability, pharmacokinetics (PK), pharmacodynamics (PD) and preliminary antitumor activity of Compound A. The study medication is administered orally either once or twice daily at escalating doses within the range from 20 mg BID to 200 mg BID. Oral administration of Compound A to patients leads to systemic exposure of Compound A and biotransformation of Compound A to Compound B by in vivo N-demethylation. For pharmacokinetic (PK) analysis, blood samples were obtained on Cycle 1, Day 15 just prior to the morning dose of Compound A and at 0.5, 1, 2, 4, 6, 8, and 10-12 hr post-dose. Compound A and its active metabolite, Compound B, were assayed using a validated bioanalytical method. Phoenix WinNonlin version 6.3 was used to analyze plasma concentration versus time data for calculation of standard noncompartmental PK parameters. All PK calculations were completed using the nominal sample collection times.

By way of exemplification, administration of Compound A to a cohort of patients at doses of 150 mg twice daily or 150 mg once daily resulted in Cycle 1 Day 15 steady state exposure to Compound A and also to Compound B as indicated in the Table below.

An oral 150 mg dose of Compound A administered BID (twice daily) to a cohort of 5 patients for 15 days afforded exposure to Compound A with a mean Cmax=1,500 ng/mL and a mean Area Under the Curve (AUC)=11,400 ng*h/mL. This 15 day dosing led to biotransformation to Compound B with a mean Cmax=1,520 ng/mL and a mean AUC=15,100 ng*h/mL. An oral 150 mg dose of Compound A administered QD (once daily) to a cohort of 4 patients for 15 days afforded exposure to Compound A with a mean Cmax=861 ng/mL and a mean Area Under the Curve (AUC)=8,070 ng*h/mL. This 15 day dosing led to biotransformation to Compound B with a mean Cmax=794 ng/mL and a mean AUC=8,600 ng*h/mL.

TABLE 1

| Oral | Compound A | Compound A | Compound B | Compound B |
|---|---|---|---|---|
| dose of | Cmax | AUC₁₂ₕ | Cmax | AUC₁₂ₕ |
| Compound A | (ng/mL) | (ng*h/mL) | (ng/mL) | (ng*h/mL) |
| 150 mg BID | 1,500 | 11,400 | 1,520 | 15,100 |
| 150 mg QD | 861 | 8,070 | 794 | 8,600 |

### EQUIVALENTS

Those skilled in the art will recognize, or be able to ascertain, using no more than routine experimentation, numerous equivalents to the specific embodiments described specifically in this disclosure. Such equivalents are intended to be encompassed in the scope of the following claims.

What is claimed is:

1. A method of treating a PDGFRα gastrointestinal stromal tumor in a patient in need thereof, comprising administering to the patient about 150 mg to about 500 mg, once or twice daily, of the compound 1-[4-bromo-5-[1-ethyl-7-(methylamino)-2-oxo-1,2-dihydro-1,6-naphthyridin-3-yl]-2-fluorophenyl]-3-phenylurea.

2. The method of claim 1, comprising administering to the patient 150 mg of the compound once daily.

3. The method of claim 1, comprising administering to the patient 150 mg of the compound twice daily.


SEQUENCE LISTING (3 sequences)

SEQ ID NO: 1 - protein, 792 aa, Homo sapiens
MEHHHHHHHHMAPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRHNQTSLYKKAGFEGDRTMKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 2 - protein, 782 aa, Homo sapiens
MAPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRHNQTSLYKKAGFEGDRTMKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARVIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 3 - protein, 802 aa, Homo sapiens
MEHHHHHHHHMAPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRHNQTSLYKKAGFEGDRTMQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDKDYIIPLPDPKPEVADEGPLEGSPSLASSTLNEVNTSSTISCDSPLEPQDEPEPEPQLELQVEPEPELEQLPDSGCPAPRAEAEDSFL